(19) United States
(12) Patent Application Publication
(10) Pub. No.: US 2009/0208984 A1
(43) Pub. Date: Aug. 20, 2009

(54) DETECTION OF FOOD SPECIFIC HUMAN IgG4 ANTIBODIES

(76) Inventors: David L. Scott, Conyers, GA (US); James Alexander Bralley, III, Johns Creek, GA (US); Robert M. David, Atlanta, GA (US); Joseph Marshall George, Sugar Hill, GA (US)

Correspondence Address: ATTN: E.J. ASBURY III;TAYLOR BUSCH, LLP, 1600 PARKWOOD CIRCLE, SUITE 200, ATLANTA, GA, 30339, US

(21) Appl. No.: 12/059,852

(22) Filed: Mar. 31, 2008

Related U.S. Application Data
(60) Provisional application No. 60/909,326, filed on Mar. 30, 2007.
(60) Provisional application No. 60/909,329, filed on Mar. 30, 2007.

(51) Int. Cl. G01N 33/53

(52) U.S. Cl. 435/7.92; 435/7.1

(57) ABSTRACT

This invention particularly discloses an improved immunoassay method for the sensitive and specific detection of food specific human IgG4 antibodies. A sample diluent comprising a chaotrophic agent is used to reduce the occurrence of nonspecific antibody-dietary antigen interactions. To reduce competition between IgE and IgG4 antibodies for specific epitopes on dietary antigens a heat denaturing step is included to inactivate IgE antibodies. Finally, a signal amplification step is included in the assay to reduce the amount of sample required to perform the assay.

### CROSS REFERENCE TO A PROVISIONAL APPLICATION

This application claims the benefit of Provisional Application Ser. No. 60/909,326, filed on Mar. 30, 2007, and Provisional Application Ser. No. 60/909,329, also filed on Mar. 30, 2007, and the entirety of each is hereby incorporated herein by reference.

### BACKGROUND OF THE INVENTION

1. Field of the Invention

The present invention generally relates to improved methods for the specific detection of food specific antibodies in biological samples.

2. Background of the Invention

Researchers estimate that at least 60% of the U.S. population suffers from unsuspected food reactions that can cause or complicate health problems. Symptoms can be extraordinarily diverse, ranging from arthritis to eczema to migraines. For that reason, many health professionals routinely consider food allergies or intolerances when evaluating a patient's health problems.

Immune-mediated adverse reactions to foods can be divided into distinct clinicopathologic entities based on presentation (immediate or delayed), target organ specificity, and pathogenic mechanisms. By far, the most common reactions are IgE mediated and dependent on activation of mast cells in specific tissues. Such reactions are immediate and in severe cases may be life-threatening. Allergic eosinophilic gastroenteritis in some instances also appears to be due to repeated and frequent IgE-mast cell-mediated reactions in the gastrointestinal mucosal (Tsai M J, J Microbiol Immunol Infect. 2000 September; 33(3):197-201). Food-induced colitis/enterocolitis is observed almost exclusively in infants and children and is not strictly IgE dependent (Lake, M. J Pediatr Gastroenterol Nutr. 2000; 30 Suppl:S58-60). Finally, gluten-sensitive enteropathy (celiac sprue) and dermatitis herpetiformis are due to abnormal immune responses to gluten (gliadin) that are non-IgE related (Ciccocioppo R, et al. Clin Exp Immunol. 2005 June; 140(3):408-16).

Non-IgE mediated food allergy and intolerance reactions have been linked to IgG antibodies (Dixon, H S. Otolarygol Head Neck Surg. 2000 July; 123 (1pt1);48-54). Non-atopic or “delayed” food reactions caused by IgG antibodies worsen or contribute to many different health problems and are considered the most common form of immunologically mediated food intolerance. These reactions are more difficult to notice since they can occur hours or even days after consumption of an offending food. In some cases, a person may eat a food for several days before developing a reaction to it, so they may not realize the link between the food and their symptoms. These “hidden” food allergies are caused by increasing blood levels of IgG antibodies in reaction to specific foods (Farenholz, J Current Treatment Options in Gastroenterology 2002, 5:39-42). Often the offenders are frequently eaten foods that are hard to avoid, such as milk, corn, and wheat.

Although there are four subclasses of IgG, IgG4 has been reported to be the predominate subclass in IgG food specific antibodies (J Allergy Clin Immunol 1995; 95:652-654). The pathophysiological processes that underlie this increase in IgG4 antibody response to common articles of food has been controversial. However, a number of reports have linked raised IgG4 antibody levels to several atopic conditions (Shakib F, et al. Int Arch Allergy Appl Immunol 1984; 75:107-112.). The role of IgG and IgA antigliadins antibodies in celiac disease is well documented. In contrast to healthy infants, in whom IgG titers decrease in time, atopic children continue to produce IgG antibodies to eggs and milk suggesting an underlying disturbance to immune regulation (Shakib F. et al. Int Arch Allergy Appl Immunol 1984; 75:107-112.) High levels of IgG antibodies have been reported in patients with eczema and/or asthma caused by milk intolerance. In a separate report high levels of IgG4 antibodies were detected in patients suffering from atopic dermatitis and/or bronchial asthma caused by hypersensitivity to soybean (Zar et al. Am J Gastroenterol 2005; 100:1500-1557). The exclusion of the offending food from the diet has shown to improve the symptoms of these conditions. Based upon these reports the detection of food-specific IgG4 is a critical prerequisite for both the definitive diagnosis and the therapeutic strategy for food intolerance disorders.

Immunological assays have been used to detect food-specific IgG antibodies. Although IgG antibody tests have been offered commercially since the 1980's, there has been little research assessing their diagnostic reliability. However, available methods for the detection of food-specific IgG antibodies have serious pitfalls that may lead to erroneous interpretations (B. Niggemann, C. Grüber Allergy Volume 59 Page). Furthermore, the measurement of food-specific IgG titers using immunological assays does not provide any information concerning the functionality of antibodies. With no established reference value as to what constitutes a harmful IgG allergic reaction, each lab uses its own criteria, which means that the results from one lab cannot be compared to that of another lab. Blinded testing of duplicate blood samples has even found that the results provided by an individual lab may not be consistent.

Further complicating matters is that the architecture of many immunoassays for the detection of IgG4 antibodies do not consider, 1) competition with coexisting IgE; 2) IgG autoantibodies to IgG4; 3) heterophillic antibodies; 4) interference with non-specific IgG; all which may impede an exact IgG4 determination in the assay (Petra et al Journal of Immunotoxicology. Volume 1, Number 3-4/2004.189-199.).

Using IgE as an example, Vassella et al. (1990) developed a method enabling the measurement of anti-IgE antibodies in free form as well as in immune complexes of IgE and anti-IgE. Anti-IgE antibodies were purified from serum of one selected blood donor with highly elevated levels of such autoantibodies. These purified anti-IgE auto-antibodies inhibited the measurement of myeloma IgE. Purification also revealed that 98% of the subject's serum IgE was masked by anti-IgE auto-antibodies. This data suggest that IgE determinations in sera containing anti-IgE antibodies might be underestimated. In such cases, false-negative results may occur due to IgG antibody competition for the same epitopes.

There are several methods available for eliminating competition interference by specific classes of human antibodies in direct ELISA (Petra et al Journal of Immunotoxicology. Volume 1, Number 3-4/2004.189-199.). One example is the capture ELISA technique, anti-IgE antibodies immobilized on ELISA microplates capture IgE in sera, before labeled allergens are added to detect allergen-specific IgE among captured IgE antibodies (Yukio et al. International Archives of Allergy and Immunology 2000; 122:264-269.). A major limitation of this method is that the immobilized anti-IgE antibodies capture all IgE regardless of their specificities. Thus another competition for immobilized competition can take place between specific IgE and nonspecific IgE, which can also lead to inaccurate determination of specific IgE.

Ammonium sulfate precipitation has also been used to remove IgG from sera (Yukio et al. International Archives of Allergy and Immunology 2000; 122:264-269.). However, the conditions for precipitation are strict, and may not be manageable, especially with a small amount of sera. Recently, protein G immobilized to solid support provided an efficient mean for IgG removal from serum (Yukio et al. International Archives of Allergy and Immunology 2000; 122:264-269.). However, this approach unexpectedly reduced detection of IgE. These results strongly suggested the presence of IgG anti-IgE autoantibodies.

Other methods for the elimination of competition between specific classes of human antibodies take advantage of differential avidities of antibodies. Antibody avidity is a measure of the binding strength between the antibody combining site and the antigenic determinant. This is an important qualitative parameter of the immune response and there is considerable evidence that high affinity antibodies are more effective than low affinity antibodies in a variety of biological reactions (Steward M W. Immunol Today 1981; July: 134-140). A tendency to produce a predominantly low affinity antibody response may result in defective antigen clearance and predisposition to immune complex disease (Devey M E, Bleadsdale K, Stanley C, et al. Immunology 1984; 52:377-383).

Determinations of the avidity of immunoglobulins are carried out in various assay systems, for example in protein-denaturing immunoassays, which are disclosed to those skilled in art in the following publications: J. Schubert et al. (1996), J. Lab. Med. 20 (12): 713-717; J. J. Gray (1995), J. Virol. Methods 52: 95-104; J. Polanec et al. (1994), J. Clin. Lab. Analysis 8: 16-21; H. O. Kangro et al. (1991), J. Med. Virol. 33: 100-105. Examples of commonly used denaturing substances are urea, diethylamines, guanidines, and thiocyanates.

There is a need in the art for an immunoassay developed for the sensitivity and specific detection of food specific human IgG4 antibodies. A assay that eliminates all or most of the false positive results generated by current assays without affecting the sensitivity of the assay. It is thus to such an improved immunoassay method for detecting food specific antibodies that the present invention is directed.

### SUMMARY OF THE INVENTION

In one aspect the present invention is a method for detecting food specific human antibodies including the steps of; providing a test sample suspected of containing food specific antibodies, contacting the test sample with specific dietary antigens in the presence of a sample diluent to form a complex of dietary antigens and human antibodies, contacting the dietary antigen human antibody complex with anti-human antibodies to form ternary complexes, contacting the ternary complex with an indicator reagent, and detecting the presence or absence of the ternary complex.

In another aspect of the present invention the dietary antigens are immobilized on a solid support. The solid support comprises at least one of a membrane, filter, piece of plastic, piece of glass, or bead. The solid support can be made from a variety of materials including; polypropylene, polystyrene, polyvinyl chloride, polyamide, polycarbonate, polyether, polymethyl methacrylate, nitrocellulose, polyvinylidene difluoride, agarose, metal, or nylon.

In yet another aspect of the present invention, the test sample is obtained from a bodily fluid. The test sample may be obtained from whole blood, from serum, or from saliva.

In yet another aspect of the present invention, the sample diluent includes a blocking reagent that can be a bovine serum albumin, casein, gelatin, milk, sucrose, or tween 20, and the sample diluent also includes a chaotrophic agent that can be a guanidine chloride, lithium chloride, or urea. The sample diluent can contain between 0.1 to 5.0% casein, 0.5 M to 4 M urea, 0.15 to 2.0 M sodium chloride, and 0.1% to 1% Tween 20. In yet another aspect of the present invention, a wash solution separates non-specific binding in the sample diluent, the wash solution comprising between about 0.1 M urea and about 0.5 M urea.

In another aspect of the present invention, the anti-human antibodies are conjugated to a detectable label. The detectable label can be a protein, an enzyme, a radioisotope, a nucleic acid segment, a fluorochrome, or biotin. In other aspects, the anti-human antibodies are specific for Total IgG human antibodies, or the anti-human antibodies are specific for IgG4 human antibodies.

In yet another aspect, a heat denaturation step is included to inactivate competing IgE antibodies for epitopes on dietary antigens. The heat denaturation step may be applied where the anti-human antibodies are specific for Total IgG human antibodies, or where the anti-human antibodies are specific for IgG4 human antibodies.

In yet another aspect of the present invention, the indicator reagent is conjugated to an enzyme. The enzyme may include horseradish peroxidase, alkaline phosphatase, or .beta.-galactosidase, or a combination thereof. The enzyme catalyzes the conversion of a non-chemiluminescent reagent into a chemiluminescent product. The enzyme enzyme may also catalyze the conversion of a non-colorimetric reagent to a calorimetric product.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### DETAILED DESCRIPTION OF PREFERRED EMBODIMENTS

The present invention provides improved immunoassays for the detection of food specific antibodies. These assays comprise contacting a test sample suspected of containing food specific antibodies with food specific antigens bound to a solid support in the presence of a blocking agent, such as casein, and a chaotrophic agent, such as urea. The present invention discloses that non-specific human antibodies may be involved in nonspecific protein-protein interactions leading to false positive assay results. These assays may further comprise the use of casein and urea in sample diluent. The incorporation of casein and urea may further reduce nonspecific binding of human antibodies to dietary antigens used in the improved immunoassay, thereby further reducing the incidence of false positive assay results. In additional preferred embodiments, kits and compositions are provided to facilitate performance of the disclosed immunoassays.

A single test that will accurately inform a physician of clinical conditions used to diagnose patients who may suffer from food allergies or food intolerance has been developed. The test utilizes a test method that measures antibody titers to food antigens. The test can also utilize a test method that measures the antibodies' ability to bind to a recombinant antigen, synthetic peptide, a peptide prepared by enzymatic digestion corresponding to the food antigen, or different cross-reactive tissue antigens.

In order to assist the physician to make a more etiologic based diagnosis, we have developed an immunoassay for detecting food allergies and food intolerance in a patient using different bodily fluids such as saliva, serum and whole blood. Surprisingly, it has been found within the scope of the present invention that the problems described above can be solved by combining blocking agents with chaotrophic agents. A number of immunoassays use chaotrophic agents such as urea to improve the qualitative or quantitative detection of an antibody. These assay bring antibodies into contact with the antigen against which it is directed so that immune complexes are able to form, then the binary complex is brought into contact with urea which destabilizes immune complexes containing antibodies of low avidity, while immune complexes containing antibodies of higher avidity are substantially retained, and in which the extent of the binding of the antibody to the antigen is determined by a method known to the skilled worker. However, this approach will not allow the recovery of IgG4 antibodies specific to dietary antigens.

IgG4 has been reported to be functionally monovalent (Aalberse and Schuurman Immunology, Volume 105, Number 1, January 2002, pp. 9-19(11). The avidity of monovalent antibodies is significantly lower than that of a corresponding bivalent molecule. Thus in the present invention a concentration of chaotrophic is use to differentiate non-specific antibody interference from low avidity IgG4 antibodies that are specific for dietary antigens. The assay eliminates all or most of the false positive results generated by current assays without affecting the sensitivity of the assay.

The assays of this invention may further comprise addition of a blocking agent to further reduce the incidence of nonspecific protein-protein interactions leading to false positive assay results. Finally the assays of this invention may include an IgE heat inactivation step to eliminate the competition between IgE and IgG4 for the same epitopes in dietary antigens. The method of the improved immunoassay typically comprises several steps, as outlined below.

### Formation of a Dietary Antigen Human Antibody Complex

The food specific antigens can be prepared from methods-well known to those of skill in the art. The food specific antigens can be immobilized on a solid support. The solid support can be provided in one of many different forms. Representative examples of solid support materials include membranes, filters, glass, plastic, plastic beads, agarose beads, SEPHAROSE™ beads (SEPHAROSE™ is a registered trademark of Pharmacia Biotech, Piscataway, N.J.), and magnetic beads.

In addition to the different forms, the solid support can be composed of a variety of materials. In one embodiment of the present invention, the solid support material may be nitrocellulose, polyvinylidene difluoride, nylon, rayon, cellulose acetate, agarose, SEPHAROSE™ metal, polypropylene, polyethylene, polystyrene, polyvinyl chloride, polyvinyl acetate, polyamide, polyimide, polycarbonate, polyether, polyester, polysulfone, polyacetal, polystyrene, or polymethyl methacrylate; in a more preferred embodiment, the material is polypropylene, polystyrene, polyvinyl chloride, polyamide, polycarbonate, polyether, polymethyl methacrylate, nitrocellulose, polyvinylidene difluoride, or nylon; and most preferably is polypropylene or polystyrene.

A test sample suspected of containing food specific antibodies is provided. The test sample is contacted with food specific antigens in the presence of a casein (blocking agent) and urea (chaotrophic agent) to reduce non-specific binding. This results in the formation of a binary complex comprising the food specific antigen and food specific human antibodies. The binary complex can be washed several times to effectively remove any uncomplexed material.

The test sample can be diluted with a sample diluent, which can comprise urea (N₂H₄CO). The incorporation of urea, a compound with chaotropic activity, reduces the nonspecific binding of antibodies to dietary antigens and/or a solid phase support material used in the assay. The urea increases the stringency of the antibody:antigen interaction, with the effect that low-avidity complexes resulting in false positive assay results are excluded. Such low avidity complexes can be composed of nonspecific antibody that has bound to cross-reactive epitopes on a food antigen. Preferably, the sample diluent will comprise between about 0.1 M and about 4 M urea, more preferably between about 0.1 M and 1 M urea, and most preferably about 0.5 M urea. To further improve the specificity of the assay casein is added to the sample diluent between about 0.1% and about 5%, more preferably about 1%. Finally, the diluted sample is heated at 56 C for 0.1-1 h to inactive IgE antibodies that may compete with IgG4 for specific epitopes on dietary antigens.

The wash buffer, or wash solution, used to wash the bound binary and/or ternary complexes described above can comprise urea. In one embodiment of the present invention, the wash buffer will preferably comprise about 0.05 M urea to about 0.5 M urea. More preferably, the wash buffer will comprise approximately 0.1 M urea. One of ordinary skill in the art is aware that these wash buffers may otherwise vary in their composition, but still be compatible with the present invention.

### Formation and Detection of Labeled Dietary Antigen Human Antibody Complexes

The dietary antigen human antibody complex is then contacted with an anti-human antibody which results in the formation of a ternary complex. The ternary complex is comprised of the food specific antigen, a food specific antibody specific for the food antigen, and the anti-human antibody. The anti-human antibody is generally conjugated to a detectable label. This ternary complex can be washed several times to remove any uncomplexed material. The ternary complex will preferably be washed prior to the detection step.

The ternary complexes can be detected either directly or with a suitable detection agent. The particular detection agent selected will depend on the type of detectable label used. A positive signal indicates the presence of food specific antibodies the test sample, thereby suggesting that the individual providing the test sample may have recently come in contact with the food product. Conversely, the absence of a signal indicates the absence of food specific antibodies in the test sample.

The test sample can generally be any biological material containing antibodies. Such materials can be processed so that they are provided in a suitable form. The test sample is preferably provided from a bodily fluid, more preferably is provided from blood, and most preferably is provided from serum or saliva.

The indicator reagent is typically conjugated to a detectable label. The detectable label can be an enzyme, such as alkaline phosphatase, .beta.-galactosidase, or peroxidase; a protein, such as biotin or digoxin; a fluorochrome, such as rhodamine, phycoerythrin, or fluorescein, or other enzyme as are known to those skilled in the art; a fluorescent protein such as GFP or one of its many modified forms as are know in the art; a radioisotope; or a nucleic acid segment.

Enzymes, such as horseradish peroxidase, alkaline phosphatase, or .beta.-galactosidase, or a combination thereof, can also be used as detectable labels. Detection agents for enzymes generally utilize a form of the enzyme's substrate. The substrate is typically modified, or provided under a set of conditions, such that a chemiluminescent, calorimetric, or fluorescent signal is observed after the enzyme and substrate have been contacted (Vargas et al., Anal. Biochem. 209: 323, 1993). Radioisotopes can alternatively be detected indirectly by autoradiography, i.e. by exposure to x-ray film.

There are many other suitable detection methods compatible with the instant invention. In each case, the detection agent and its method of use are well known to one of ordinary skill in the art.

A diagnostic kit can be designed to aid in the performance of the above method. Such a kit can contain food specific antigens affixed to a solid support and the indicator reagent, respectively. The kit can further contain test sample diluent, various blocking buffers, buffers to aid the formation of binary and ternary complexes, wash buffers, and detection agents. One of ordinary skill in the art is aware that the diluent and buffers can vary in their exact composition, but still be compatible with the present invention. All such variations are considered equivalent for the purposes of the present invention. The test sample diluent and/or wash buffers can comprise urea, as described above.

The diagnostic kit can further comprise a detection agent. As previously mentioned, the choice of a suitable detection agent generally depends on which detectable label is used. Many different detectable labels and detection agents are compatible with the present invention and exemplary embodiments follow.

The components of the diagnostic kit can be provided in many different forms and quantities. Various types of packaging can also be used. Instructions for the correct use of the kit can be supplied with the kit. Any such alternative embodiments are considered equivalent to the present invention.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of the Solid Support Material

30 different dietary antigens were assayed in an antigen-dilution IgG4 ELISA to determine the optimal antigen concentration for sensitization of the solid phase. The dietary antigens were diluted in bicarbonate-carbonate buffer, pH 9.5, to within the range of 0.1 μg/ml to 1000 μg/ml, typically 100 μg/ml, and 0.1 mL was placed in each well of a 96-well microtiter plate. The plates were incubated at 4-7° C. overnight to allow the glycoproteins to adsorb to the plastic surface. After adsorption, the fluid was removed from the plate wells. A blocking solution of phosphate buffered saline, pH 7.0 containing 1% w/v casein (0.2 mL) was then added to each well and the plates were incubated overnight at 4-7° C. The blocking solution contained proteins that adsorb to the remaining sites on the plastic wells and help block subsequent adsorption of the test serum globulins to the plastic surface. After blocking, the fluid was removed from the plate wells and the plates were allowed to air dry. The plates were then sealed in a foil pouch along with a dessicant bag.

### Example 2

### Preparation of the IgG4 Serum Diluent

### Buffer 1

Phosphate buffered saline plus 0.05% Tween and 1% Casein consists of 8.0 gm of NaCl, 2.9 gm of Na2HPO4, 0.2 gm of KH2PO4, 0.2 gm of KCl, 0.5 ml of Tween 20, and 10 g of casein in 1 liter of purified water, pH is 7.4.

### Buffer 2

Phosphate buffered saline plus 0.05% Tween and 1% Casein consists of 8.0 gm of NaCl, 2.9 gm of Na2HPO4, 0.2 gm of KH2PO4, 0.2 gm of KCl, 0.5 ml of Tween 20, 10 g of casein, and 30.03 g urea in 1 liter of purified water, pH is 7.4.

### Buffer 3

Phosphate buffered saline plus 1.0% Tween consists of 29.22 gm of NaCl, 2.9 gm of Na2HPO4, 0.2 gm of KH2PO4, 0.2 gm of KCl, and 10.0 ml of Tween 20 in 1 liter of purified water, pH is 7.4.

### Example 3

### Detection of Food Specific IgG4 in Serum

The serum test samples, an IgG4 calibrator serum and an IgG4 positive control serum were individually diluted 1:10 or greater in Diluent buffer 2. The dilutions were allowed to incubate at room temperature for 10 to 60 minutes, after which 0.1 ml of each diluted serum was placed in a separate well of the antigen-coated plate described in Example 1. After addition of all the serum samples, the plate was incubated at room temperature for 1 h. The fluid was removed by inverting the plate over a sink or beaker and then slapping the plate on paper towels to remove any excess diluted serum. Each well was washed three times with a wash buffer consisting of phosphate buffered saline containing 0.05% (vol/vol) Tween™ 20 detergent (Tween™ is a registered trademark of Robin and Haas Co., Spring House, Pa.). The wells were filled with wash buffer and the fluid was removed as described above. After the final wash was removed from the wells, 0.1 ml of a murine monoclonal antihuman IgG4 conjugated with biotin obtained from Sigma (St. Louis, Mo.) was placed in each well. The anti-IgG4 conjugate was diluted 1:50,000 in sample diluent 2. The conjugate was allowed to incubate for 30 minutes at room temperature. After incubation, the conjugate was moved from the wells, and each well was washed 3 times with wash buffer as described above. After the last wash was removed from the wells, 0.1 ml of Bcon100™ Reporter Molecule (Peroxidase) (Singulus Molecular, Conyers, Ga.) was added to each well and incubated for 15 min at room temperature. After incubation, the Bcon100™ Reporter Molecule (Peroxidase) was moved from the wells, and each well was washed 3 times with wash buffer as described above. After the last wash was removed from the wells, 0.1 ml SureBlue TMB 1-Component Microwell Peroxidase Substrate (KPL, Gaithersburg, Md.) solution was placed in each well, and the plate was incubated for 20 minutes at room temperature in a moist chamber to allow color development. The color was read at 450 nm using a spectrophotometer.

The test absorbance values were assigned quantitative values (ng/ml) by the following method:

(i) obtain the absorbance value for the blank well(s) containing everything but the serum;
(ii) obtain absorbance values for test and IgG4 calibrator samples;
(iii) subtract the blank absorbance value from each of the test and calibrator sample values to obtain corrected values, if a test sample value is less than the cut-off point, which is two standard deviations above the mean blank wells absorbance, it is corrected to zero; and
(iv) plot absorbances (Y-axis) of calibrators versus the calibrator concentrations (X-axis);
(v) determine the IgG4 concentration of each test sample by interpolating from the standard curve or by using linear regression analysis;
(vi) If a specimens value exceeds the limits of the standard curve, dilute the specimen and retest; otherwise
(vii) multiply the IgG4 concentration in the test sample by the dilution factor (dilution factor is the ratio of serum to sample diluent to provide quantity of IgG4 expressed as ng/ml.

### Example 4

### Comparison of Different Urea Concentrations in Sample Diluent in New IgG4 ELISA

A patient serum that is used to QC the present “in house” IgG4 ELISA was tested using the new method employing a panel of 30 dietary antigens. The serum test samples, an IgG4 calibrator serum and an IgG4 positive control serum were individually diluted 1:40 in sample diluent 1 containing 1% 0.0 M, 0.5 M, 1.0 M, 2.0 M, 3.0 M urea respectively. The dilutions were allowed to incubate at room temperature for 10 to 60 minutes, after which 0.1 ml of each diluted serum was placed in a separate well of the antigen-coated plates and food specific IgG4 human antibodies were detected as described in Example 3. As seen in Table 1 when the assay was performed, with urea omitted from the sample diluent, the assay detected IgG4 specific antibodies for almond, cashew, egg, milk, mustard, soybean, sunflower, and tuna. The concentrations of these specific antibodies ranged from 109 ng/ml to 312 ng/ml.

The same sample was tested using the new method as described in example 3 with the inclusion of increasing concentrations of urea in the sample diluent. As seen in Table 2 when a sample diluent containing 0.5 M urea was used the assay detected IgG4 specific antibodies for almond, cashew, egg, mustard, and sunflower. As the concentration of urea was increased the concentration of IgG4 food specific antibodies decreased (Compare dietary antigens in Tables 1-Table 5). Only IgG4 specific antibodies for cashew and mustard were still quantifiable at 3M urea demonstrating strong avidity. These results indicates that the new method can classify IgG4 antibodies interaction with dietary antigens into 3 categories i. non-specific; ii. Low avidity; iii. High avidity.

### Example 5

### Detection of Food Specific IgG4 in Whole Blood (Bloodspot)

Bloodspots (1-4) were cut out and diluted in 3.5 ml of diluent buffer 3. The cut-out spots were shaked for 60 minutes, after which 0.1 ml of each diluted serum was placed in a separate well of the antigen-coated plate described in Example 2. After addition of all the serum samples, the plate was incubated at room temperature for 1 h. The fluid was removed by inverting the plate over a sink or beaker and then slapping the plate on paper towels to remove any excess diluted serum. Each well was washed three times with a wash buffer consisting of phosphate buffered saline containing 0.05% (vol/vol) Tween™ 20 detergent (Tween™. is a registered trademark of Robin and Haas Co., Spring House, Pa.). The wells were filled with wash buffer and the fluid was removed as described above. After the final wash was removed from the wells, 0.1 ml of a murine monoclonal antihuman IgG4 conjugated with biotin obtained from Sigma (St. Louis, Mo.) was placed in each well. The anti-IgG4 conjugate was diluted 1:50,000 in phosphate buffered saline containing 1% (wt/vol) casein prior to use. The conjugate was allowed to incubate for 30 minutes at room temperature. After incubation, the conjugate was moved from the wells, and each well was washed 3 times with wash buffer as described above. After the last wash was removed from the wells, 0.1 ml of Bcon100™ Reporter Molecule (Peroxidase) (Singulus Molecular, Conyers, Ga.) was added to each well and incubated for 15 min at room temperature. After incubation, the Bcon100™ Reporter Molecule (Peroxidase) was moved from the wells, and each well was washed 3 times with wash buffer as described above. After the last wash was removed from the wells, 0.1 ml SureBlue TMB 1-Component Microwell Peroxidase Substrate (KPL, Gaithersburg, Md.) solution was placed in each well, and the plate was incubated for 20 minutes at room temperature in a moist chamber to allow color development. The color was read at 450 nm using a spectrophotometer.

The test absorbance values were assigned quantitative values (ng/ml) by the following method:

(i) obtain the absorbance value for the blank well(s) containing everything but the serum;
(ii) obtain absorbance values for test and IgG4 calibrator samples;
(iii) subtract the blank absorbance value from each of the test and calibrator sample values to obtain corrected values, if a test sample value is less than the cut-off point, which is two standard deviations above the mean blank wells absorbance, it is corrected to zero; and
(iv) plot absorbances (Y-axis) of calibrators versus the calibrator concentrations (X-axis);
(v) determine the IgG4 concentration of each test sample by interpolating from the standard curve or by using linear regression analysis;
(vi) If a specimens value exceeds the limits of the standard curve, dilute the specimen and retest; otherwise
(vii) multiply the IgG4 concentration in the test sample by the dilution factor (dilution factor is the ratio of serum to sample diluent to provide quantity of IgG4 expressed as ng/ml.

### Example 6

### Detection of Food Specific IgG4 in Saliva

The serum test samples, an IgG4 calibrator serum and an IgG4 positive control serum were individually diluted 1:10 or greater in sample diluent 2. The dilutions were allowed to incubate at room temperature for 10 to 60 minutes, after which 0.1 ml of each diluted serum was placed in a separate well of the antigen-coated plate described in Example 2. After addition of all the serum samples, the plate was incubated at room temperature for 1 h. The fluid was removed by inverting the plate over a sink or beaker and then slapping the plate on paper towels to remove any excess diluted serum. Each well was washed three times with a wash buffer consisting of phosphate buffered saline containing 0.05% (vol/vol) Tween™0.20 detergent ( ) Tween™ is a registered trademark of Robin and Haas Co., Spring House, Pa.). The wells were filled with wash buffer and the fluid was removed as described above. After the final wash was removed from the wells, 0.1 ml of a murine monoclonal antihuman IgG4 conjugated with biotin obtained from Sigma (St. Louis, Mo.) was placed in each well. The anti-IgG4 conjugate was diluted 1:50,000 in phosphate buffered saline containing 1% (wt/vol) casein prior to use. The conjugate was allowed to incubate for 30 minutes at room temperature. After incubation, the conjugate was moved from the wells, and each well was washed 3 times with wash buffer as described above. After the last wash was removed from the wells, 0.1 ml of Bcon100™ Reporter Molecule (Peroxidase) (Singulus Molecular, Conyers, Ga.) was added to each well and incubated for 15 min at room temperature. After incubation, the Bcon100™ Reporter Molecule (Peroxidase) was moved from the wells, and each well was washed 3 times with wash buffer as described above. After the last wash was removed from the wells, 0.1 ml SureBlue TMB 1-Component Microwell Peroxidase Substrate (KPL, Gaithersburg, Md.) solution was placed in each well, and the plate was incubated for 20 minutes at room temperature in a moist chamber to allow color development. The color was read at 450 nm using a spectrophotometer.

The test absorbance values were assigned quantitative values (ng/ml) by the following method:

(i) obtain the absorbance value for the blank well(s) containing everything but the serum;
(ii) obtain absorbance values for test and IgG4 calibrator samples;
(iii) subtract the blank absorbance value from each of the test and calibrator sample values to obtain corrected values, if a test sample value is less than the cut-off point, which is two standard deviations above the mean blank wells absorbance, it is corrected to zero; and
(iv) plot absorbances (Y-axis) of calibrators versus the calibrator concentrations (X-axis);
(v) determine the IgG4 concentration of each test sample by interpolating from the standard curve or by using linear regression analysis;
(vi) If a specimens value exceeds the limits of the standard curve, dilute the specimen and retest; otherwise
(vii) multiply the IgG4 concentration in the test sample by the dilution factor (dilution factor is the ratio of serum to sample diluent to provide quantity of IgG4 expressed as ng/ml.

TABLE 1
Quantification (ng/ml) of food specific IgG4
antibodies using new IgG4 food-specific ELISA

| Beef | Crab | Rice | Sunflower | Corn |
|---|---|---|---|---|
| 0 | 0 | 0 | 0 | 0 |

| Chicken | Salmon | Wheat | Walnut | Almond |
|---|---|---|---|---|
| 0 | 0 | 0 | 0 | 139 |

| Egg | Shrimp | Oat | Cashew |
|---|---|---|---|
| 210 | 0 | 0 | 312 |

| Milk | Lobster | Peas, Green | Garlic |
|---|---|---|---|
| 171 | 0 | 0 | 0 |

| Pork | Cantaloupe | Peanut | Mustard |
|---|---|---|---|
| 0 | 0 | 109 | 121 |

| Turkey | Orange | Pinto Bean | Tomato |
|---|---|---|---|
| 0 | 0 | 0 | 0 |

| Tuna | Strawberry | Soybean | Aspergillius |
|---|---|---|---|
| 252.4323 | 0 | 93.6103 | 0 |

TABLE 2
Quantification (ng/ml) of food specific IgG4 antibodies
using new IgG4 food-specific ELISA using sample
diluent 1 modified with 0.5 M urea.

| Beef | Crab | Rice | Sunflower | Corn |
|---|---|---|---|---|
| 0 | 0 | 0 | 92 | 0 |

| Chicken | Salmon | Wheat | Walnut | Almond |
|---|---|---|---|---|
| 0 | 0 | 0 | 0 | 112 |

| Egg | Shrimp | Oat | Cashew |
|---|---|---|---|
| 208 | 0 | 0 | 227 |

| Milk | Lobster | Peas, Green | Garlic |
|---|---|---|---|
| 0 | 0 | 0 | 0 |

| Pork | Cantaloupe | Peanut | Mustard |
|---|---|---|---|
| 0 | 0 | 86 | 93 |

| Turkey | Orange | Pinto Bean | Tomato |
|---|---|---|---|
| 0 | 0 | 0 | 0 |

| Tuna | Strawberry | Soybean | Aspergillius |
|---|---|---|---|
| 0 | 0 | 0 | 0 |

TABLE 3
Quantification (ng/ml) of food specific IgG4 antibodies
using new IgG4 food-specific ELISA using sample
diluent 1 modified with 1.0 M urea.

| Beef | Crab | Rice | Sunflower | Corn |
|---|---|---|---|---|
| 0 | 0 | 0 | 0 | 0 |

| Chicken | Salmon | Wheat | Walnut | Almond |
|---|---|---|---|---|
| 0 | 0 | 0 | 0 | 0 |

| Egg | Shrimp | Oat | Cashew |
|---|---|---|---|
| 154 | 0 | 0 | 166 |

| Milk | Lobster | Peas, Green | Garlic |
|---|---|---|---|
| 0 | 0 | 0 | 0 |

| Pork | Cantaloupe | Peanut | Mustard |
|---|---|---|---|
| 0 | 0 | 0 | 141 |

| Turkey | Orange | Pinto Bean | Tomato |
|---|---|---|---|
| 0 | 0 | 0 | 0 |

| Tuna | Strawberry | Soybean | Aspergillius |
|---|---|---|---|
| 0 | 0 | 0 | 0 |

TABLE 4
Quantification (ng/ml) of food specific IgG4 antibodies
using new IgG4 food-specific ELISA using sample
diluent 1 modified with 2.0 M urea.

| Beef | Crab | Rice | Sunflower | Corn |
|---|---|---|---|---|
| 0 | 0 | 0 | 0 | 0 |

| Chicken | Salmon | Wheat | Walnut | Almond |
|---|---|---|---|---|
| 0 | 0 | 0 | 0 | 0 |

| Egg | Shrimp | Oat | Cashew |
|---|---|---|---|
| 87 | 0 | 0 | 135 |

| Milk | Lobster | Peas, Green | Garlic |
|---|---|---|---|
| 0 | 0 | 0 | 0 |

| Pork | Cantaloupe | Peanut | Mustard |
|---|---|---|---|
| 0 | 0 | 0 | 147 |

| Turkey | Orange | Pinto Bean | Tomato |
|---|---|---|---|
| 0 | 0 | 0 | 0 |

| Tuna | Strawberry | Soybean | Aspergillius |
|---|---|---|---|
| 0 | 0 | 0 | 0 |

TABLE 5
Quantification (ng/ml) of food specific IgG4 antibodies
using new IgG4 food-specific ELISA using sample
diluent 1 modified with 3.0 M urea.

| Beef | Crab | Rice | Sunflower | Corn |
|---|---|---|---|---|
| 0 | 0 | 0 | 0 | 0 |

| Chicken | Salmon | Wheat | Walnut | Almond |
|---|---|---|---|---|
| 0 | 0 | 0 | 0 | 0 |

| Egg | Shrimp | Oat | Cashew |
|---|---|---|---|
| 0 | 0 | 0 | 86 |

| Milk | Lobster | Peas, Green | Garlic |
|---|---|---|---|
| 0 | 0 | 0 | 0 |

| Pork | Cantaloupe | Peanut | Mustard |
|---|---|---|---|
| 0 | 0 | 0 | 102 |

| Turkey | Orange | Pinto Bean | Tomato |
|---|---|---|---|
| 0 | 0 | 0 | 0 |

| Tuna | Strawberry | Soybean | Aspergillius |
|---|---|---|---|
| 0 | 0 | 0 | 0 |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention.

What is claimed is:

1. A method for detecting food specific human antibodies comprising:
a) providing a test sample suspected of containing food specific antibodies;
b) contacting the test sample with specific dietary antigens in the presence of a sample diluent to form a complex of dietary antigens and human antibodies;
c) contacting the dietary antigen human antibody complex with anti-human antibodies to form ternary complexes;
d) contacting the ternary complex with an indicator reagent; and
e) detecting the presence or absence of the ternary complex.

2. The method of claim 1, wherein the dietary antigens are immobilized on a solid support.

3. The method of claim 2, wherein the solid support comprises at least one of a membrane, filter, piece of plastic, piece of glass, or bead.

4. The method of claim 2, wherein the solid support is made of a material comprising at least one of a polypropylene, polystyrene, polyvinyl chloride, polyamide, polycarbonate, polyether, polymethyl methacrylate, nitrocellulose, polyvinylidene difluoride, agarose, metal, or nylon.

5. The method of claim 1, wherein the test sample is obtained from a bodily fluid.

6. The method of claim 5, wherein the test sample is obtained from whole blood.

7. The method of claim 5, wherein the test sample is obtained from serum.

8. The method of claim 5, wherein the test sample is obtained from saliva.

9. The method of claim 1, wherein the sample diluent comprises:
a) a blocking reagent that is at least one of a bovine serum albumin, casein, gelatin, milk, sucrose, or tween 20, and
b) a chaotrophic agent that is at least one of a guanidine chloride, lithium chloride, or urea.

10. The method of claim 9, wherein the sample diluent comprises between 0.1 to 5.0% casein, 0.5 M to 4 M urea, 0.15 to 2.0 M sodium chloride, and 0.1% to 1% Tween 20.

11. The method of claim 1, wherein a wash solution separates non-specific binding, the wash solution comprising between about 0.1 M urea and about 0.5 M urea.

12. The method of claim 1, 10, or 11, wherein the anti-human antibodies are conjugated to a detectable label.

13. The method of claim 12, wherein the detectable label comprises at least one of a protein, enzyme, radioisotope, nucleic acid segment, fluorochrome, or biotin.

14. The method of claim 13, wherein the detectable label is biotin.

15. The method of claims 1, 10 or 11 wherein the anti-human antibodies are specific for Total IgG human antibodies.

16. The method of claims 1, 10, or 11, wherein the anti-human antibodies are specific for IgG4 human antibodies.

17. The method of claim 1, wherein a heat denaturation step is included to inactivate competing IgE antibodies for epitopes on dietary antigens.

18. The method of claim 17 wherein the anti-human antibodies are specific for Total IgG human antibodies.

19. The method of claim 17 wherein the anti-human antibodies are specific for IgG4 human antibodies.

20. The method of claims 1, 10, 11, or 17 wherein the indicator reagent is conjugated to an enzyme.

21. The method according to claim 20, wherein the enzyme comprises at least one of horseradish peroxidase, alkaline phosphatase, or .beta.-galactosidase.

22. The method of claim 20, wherein the enzyme catalyzes the conversion of a non-chemiluminescent reagent into a chemiluminescent product.

23. The method of claim 20, wherein the enzyme catalyzes the conversion of a non-colorimetric reagent to a calorimetric product.
